(19) United States
(12) Patent Application Publication
(10) Pub. No.: US 2010/0015676 A1
(43) Pub. Date: Jan. 21, 2010

(54) METHOD FOR PRODUCING POLYESTEROLS

(75) Inventors: Mirko Kreitschmann, Mannheim (DE); Maxium Peretolchin, Mannheim (DE); Lionel Gehringer, Scheibenhard (FR); Jean-Francois Stumbe, Strasbourg (FR); Dietmar Haering, Neu-Edingen (DE); Ulrike Mahn, Mannheim (DE); Joern Duwenhorst, Lemfoerde (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/439,375

(22) PCT Filed: Aug. 29, 2007
(86) PCT No.: PCT/EP07/58983

§ 371 Date: Feb. 27, 2009

(30) Foreign Application Priority Data
Aug. 30, 2006 (EP) 06119830.5

(51) Int. Cl. C12P 7/02; C12M 1/40

(52) U.S. Cl. 435/155; 435/289.1

(57) ABSTRACT

The invention relates to a process for preparing polyesterols from at least one base polyesterol and at least one further reagent, wherein
(a) the at least one base polyesterol and the at least one further reagent are mixed,
(b) the mixture produced in (a) flows through a reactor in which at least one packing comprising at least one immobilized enzyme on a support is present, with the at least one base polyesterol and the at least one further reagent being reacted to form the polyesterol.

The present invention relates to a process for preparing polyesterols which are different from at least one base polyesterol from the at least one base polyesterol and at least one further reagent, wherein:
(a) the at least one base polyesterol and the at least one further reagent are mixed,
(b) the mixture produced in a) flows through a reactor in which at least one packing comprising at least one immobilized enzyme on a support is present.

Polymeric hydroxyl compounds such as polyesterols and polyetherols react with isocyanates to form polyurethanes which have a wide range of possible uses, depending on their specific mechanical properties. Polyesterols in particular are used for high-quality polyurethane products because of their favorable properties. The specific properties of the polyurethanes concerned depend strongly on the polyesterols used.

To produce polyurethanes, it is particularly important that the polyesterols used have a low acid number (cf. Ullmann's Encyclopedia, Electronic Release, Wiley-VCH-Verlag GmbH, Weinheim, 2000, under the keyword “Polyesters”, paragraph 2.3 “Quality Specifications and Testing”). The acid number should be very small since terminal acid groups react more slowly with diisocyanates than do terminal hydroxyl groups. Polyesterols having high acid numbers therefore lead to a lower buildup of the molecular weight during the reaction of polyesterols with isocyanates to form polyurethane.

A further problem associated with the use of polyesterols having high acid numbers for the polyurethane reaction is that the reaction of the numerous terminal acid groups with isocyanates forms an amide bond with liberation of carbon dioxide. The gaseous carbon dioxide can then lead to undesirable bubble formation. Furthermore, free carboxyl groups adversely affect the catalysis in the polyurethane reaction and also the stability of the polyurethanes produced toward hydrolysis.

On the basis of their chemical structure, polyesterols, i.e. polyesters having at least two terminal OH groups, can be divided into two groups, viz. the hydroxycarboxylic acid types (AB polyesters) and the dihydroxydicarboxylic acid types (AA-BB polyesters). The former are prepared from only one monomer by, for example, polycondensation of an ω-hydroxycarboxylic acid or by ring-opening polymerization of cyclic esters, known as lactones. On the other hand, AA-BB polyester types are prepared by polycondensation of two complementary monomers, generally by reaction of polyfunctional polyhydroxyl compounds (e.g. diols or polyols) with dicarboxylic acids (e.g. adipic acid or terephthalic acid).

The polycondensation of polyfunctional polyhydroxyl compounds and dicarboxylic acids to form polyesterols of the AA-BB type is generally carried out industrially at high temperatures of 160-280° C. The polycondensation reactions can be carried out either in the presence or absence of a solvent. However, a disadvantage of these polycondensations at high temperatures is that they proceed comparatively slowly. For this reason, esterification catalysts are frequently used to accelerate the polycondensation reaction at high temperatures. Classic esterification catalysts employed are preferably organic metal compounds, e.g. titanium tetrabutylate, tin dioctoate or dibutyltin dilaurate, or acids such as sulfuric acid, p-toluenesulfonic acid or bases such as potassium hydroxide or sodium methoxide. These esterification catalysts are homogeneous and generally remain in the polyesterol after the reaction is complete. A disadvantage of this is that the esterification catalysts remaining in the polyesterol may adversely affect the later conversion of these polyesterols into the polyurethane.

A further disadvantage is the fact that by-products are frequently formed in the polycondensation reaction at high temperatures. Furthermore, the high-temperature polycondensations have to take place with exclusion of water in order to avoid the reverse reaction. This is generally achieved by the condensation being carried out under reduced pressure, under an inert gas atmosphere or in the presence of an entraining gas for the complete removal of the water.

Overall, the reaction conditions required, in particular the high reaction temperatures, the possible inert conditions or carrying out the reaction under reduced pressure and also the necessity of a catalyst lead to very high capital and operating costs for the high-temperature polycondensation.

To avoid these numerous disadvantages of the catalyzed condensation processes, alternative processes for preparing polyesterols in which enzymes are used at low temperatures in place of esterification catalysts at high temperatures have been developed. Enzymes used are generally lipases, including the lipases Candida antarctica, Candida cylinderacea, Mucor miehei, Pseudomonas cepacia, Pseudomonas fluorescens.

In the known enzyme-catalyzed processes for preparing polyesterols of the AA-BB type, either “activated dicarboxylic acid components”, e.g. in the form of dicarboxylic acid diesters (cf. Wallace et al., J. Polym. Sci., Part A: Polym. Chem., 27 (1989), 3271) or “unactivated dicarboxylic acids” are used together with polyfunctional hydroxyl compounds. These enzymatic processes, too, can be carried out either in the presence or in the absence of a solvent.

Thus, for example, EP 0 670 906 B1 discloses a lipase-catalyzed process for preparing polyesterols of the AA-BB type at 10-90° C., which makes do without use of a solvent. In this process, it is possible to use either activated or unactivated dicarboxylic acid components.

Uyama et al., Polym. J., Vol. 32, No. 5, 440-443 (2000), also describe a process for preparing aliphatic polyesters from unactivated dicarboxylic acids and glycols (sebacic acid and 1,4-butanediol) in a solvent-free system with the aid of the lipase Candida antarctica.

Binns et al., J. Polym. Sci., Part A: Polym. Chem., 36 2069-1080 (1998) disclose processes for preparing polyesterols from adipic acid and 1,4-butanediol with the aid of the immobilized form of the lipase B from Candida antarctica (commercially available as Novozym 435®). In particular, the influence of the presence or absence of a solvent (in this case toluene) on the reaction mechanism was analyzed. It was able to be observed that the polyesterol is essentially extended only by stepwise condensation of further monomer units onto it in the absence of a solvent, while in the presence of toluene as solvent, transesterification reactions also play a role in addition to the stepwise formation of further ester links. Thus, the enzyme specificity of the lipase used appears to depend, inter alia, on the presence and type of the solvent.

However, the high-temperature polycondensations and the enzymatically catalyzed polycondensations for preparing polyesterols both have the disadvantage that the preparation of polyesterols by condensation reactions is carried out in plants for which a complicated periphery is necessary. In addition, the reaction is carried out in batch reactors, so that continuous preparation of the polyesterols is likewise not possible.

In the case of the stirred tank reactors known from the prior art, it has also been found that high catalyst concentrations exceeding 10% by weight in combination with the relatively high viscosities associated with the polymers are difficult to manage. In particular, filtration of the enzymes from the polymer is a great technical challenge since a high pressure drop is necessary because of the small size of the enzyme particles (0.3-0.5 mm), so that relatively high pressures and accordingly high-pressure reactors are necessary. Relatively high shear forces which occur as a result of relatively high viscosities lead to a relatively high stress on the immobilized enzymes, which leads to abrasion and as a result in a decrease in the life of the enzymes.

The use of continuous reactors is known from the preparation of short-chain esters. These are generally fixed-bed reactors in which the enzyme used for catalysis is immobilized on a support present in the reactor. Such reactors are, for example, used for preparing isoamyl propionate and water from propionic acid and isoamyl alcohol as in P. Mensah and G. Carta, Biotechnology and Bioengineering, Vol. 66, No. 3, 1999, 137 to 146.

A further reaction in which continuous reactors are used is the transesterification of geraniol with ethyl caproate to form geranyl caproate. This is carried out using a miniature reactor in which an enzyme immobilized on a support is present. This reaction is described by D. Pirozzi and P. J. Halling, Biotechnology and Bioengineering, Vol. 72, No. 2, 2001, 244 to 248.

Furthermore, the use of continuous reactors is also known in reactions for the degradation of biodegradable polyesters. Here, a reactor which comprises a packing comprising an enzyme present on an immobilized support is used. The polymer to be degraded is firstly dissolved in a solvent and subsequently passed through the reactor. In the reactor, the polyester is converted into cyclic oligomers. The reaction is described by Y. Osanai et al., Macromolecular Bioscience, 2004, 4, 936 to 942.

In all these reactions in which a continuous reactor is used, a readily flowable mixture leaves the reactor.

However, high molecular weight reaction products which have a varying molecular weight and can, depending on their composition, be solid or have a very high viscosity and therefore do not flow well are produced in the preparation of polyesterols.

It is an object of the present invention to provide a process by means of which polyesterols can be prepared continuously.

This object is achieved by a process for preparing polyesterols which are different from the base polyesterols from at least one base polyesterol and at least one further reagent, wherein:
(a) the at least one base polyesterol and the at least one further reagent are mixed, and
(b) the mixture produced in a) flows through a reactor in which at least one packing comprising at least one immobilized enzyme on a support is present.

In the reactor, the base polyesterol is generally converted by means of an enzymatically catalyzed transesterification reaction into the polyesterol which is different from the base polyesterol.

After the reaction, the polyesterol is liquid, in particular at the process temperature. However, some polyesterols can crystallize out on cooling.

The base polyesterol used in the reaction is, for example, prepared by polycondensation of polyhydroxy compounds and polycarboxylic acids with elimination of water, in which an excess of polyhydroxy compounds is required. The base polyesterol can here be prepared by, for example, standard methods, preferably by means of high-temperature polycondensation, more preferably by means of high-temperature polycondensation aided by an esterification catalyst.

As an alternative, it is also possible to prepare the base polyesterol by means of an enzymatic polycondensation instead of a high-temperature polycondensation aided by an esterification catalyst. In the enzymatic polycondensation, preference is given to using a lipase or hydrolase, preferably a lipase, in particular one of the lipases Candida antarctica, Candida cylinderacea, Mucor miehei, Pseudomonas cepacia, Pseudomonas fluorescens and Burkholderia plantarii, at from 20 to 120° C., preferably from 50 to 90° C. The enzymes can also be immobilized on a support material.

If a high-temperature polycondensation is carried out, an organic metal compound, for example titanium tetrabutoxide, tin dioctoate or dibutyltin dilaurate, or an acid, for example sulfuric acid, p-toluenesulfonic acid, or a base, for example potassium hydroxide or sodium methoxide, is preferably used as esterification catalyst. This esterification catalyst is generally homogeneous and generally remains in the polyesterol after the reaction is complete. The high-temperature polycondensation is carried out at from 160 to 280° C., preferably from 200 to 250° C.

In the preparation of the base polyesterol by means of a conventional high-temperature polycondensation or by means of an enzymatic polycondensation, the water liberated in the condensation reaction is preferably removed continuously.

As polycarboxylic acid, in particular dicarboxylic acid, preference is given to using adipic acid or other aliphatic dicarboxylic acids, terephthalic acid or other aromatic dicarboxylic acids. Suitable polyhydroxyl compounds are all at least dihydric alcohols, but preferably diol components such as ethylene glycol, diethylene glycol, 1,3-propanediol, 1,2-propanediol, 1,4-butanediol, 1,5-pentanediol, 1,6-hexanediol, 2-methyl-1,3-propanediol, 3-methyl-1,5-pentanediol.

The polycondensation can be carried out either in the presence of a solvent or else in the absence of a solvent, i.e. in bulk, regardless of whether a high-temperature polycondensation (aided by means of an esterification catalyst) or an enzymatically catalyzed polycondensation is carried out. However, preference is given to carrying out the polycondensation for preparing the base polyesterol in bulk, i.e. in the absence of any solvent.

The base polyesterols are chosen according to the desired properties of the end products. Base polyesterols which are preferably used are polyesterols based on adipic acid and a diol component, preferably ethylene glycol, diethylene glycol, 1,3-propanediol, 1,2-propanediol, 1,4-butanediol, 1,5-pentanediol, 1,6-hexanediol, 2-methyl-1,3-propanediol, 3-methyl-1,5-pentanediol.

The preferred molecular weight of the base polyesterols prepared by the polycondensation is in the range from 200 g/mol to 10 000 g/mol, particularly preferably in the range from 500 to 5000 g/mol.

The acid numbers of the base polyesterols prepared by the polycondensation are preferably in the range below 3 g KOH/kg, more preferably in the range below 2 g KOH/kg, in particular in the range below 1 g KOH/kg. The acid number serves to indicate the content of free organic acid groups in the polyesterol. The acid number is determined by the number of mg of KOH (or g of KOH) consumed in the neutralization of 1 g (or 1 kg) of the sample.

The functionality of the base polyesterols prepared by the polycondensation is preferably in the range from at least 1.9 to 4.0, more preferably in the range from 2.0 to 3.0. The hydroxyl number (hereinafter referred to as OHN for short) of the base polyesterols prepared by the polycondensation is calculated from the number average molecular weight Mₙ and the functionality f of the polyesterol according to the formula

$$OHN = \frac{56100 \cdot f}{{M}_{n}} .$$

It has been found that the process of the invention for preparing polyesterols, in which a base polyesterol as described above is used, can also be employed for base polyesterols which originate from classic high-temperature catalysis and thus already have a relatively high mean molecular weight (for example 3000 g/mol) and consequently also low acid numbers. It has long been known that polyesterols which have high mean molecular weights and consequently low acid numbers, in particular, have little tendency if any to undergo transesterification (cf. 2^{nd} section by McCabe and Taylor, Tetrahedon 60 (2004), 765 to 770).

The further reagent which is mixed with the base polyesterol in step a) is, for example, a further polyesterol, a polyol, an organic acid or an oligomer or polymer having at least one hydroxyl or carboxylic acid radical.

If the further reagent is a further polyesterol, this can likewise be prepared as described above.

Suitable polyols, in particular diols, which can be mixed as further reagent with the base polyesterol are, for example, ethylene glycol, diethylene glycol, 1,3-propanediol, 1,2-propanediol, 1,4-butanediol, 1,5-pentanediol, 1,6-hexanediol, 2-methyl-1,3-propanediol, 3-methyl-1,5-pentanediol, neopentyl glycol, propylene glycol, trimethylolpropane, pentaerythritol, glycerol, diglycerol, dimethylolpropane, dipentaerythritol, sorbitol, sucrose or other sugars.

Suitable organic acids which can be used as further reagent are, for example, oxalic acid, malonic acid, succinic acid, glutaric acid, adipic acid, pimelic acid, suberic acid, sebacic acid, maleic acid, oleic acid, phthalic acid, terephthalic acid.

Suitable oligomers or polymers having at least one hydroxyl or carboxylic acid radical are, for example, polytetrahydrofuran, polylactone, polyglycerols, polyetherol, polyesterol, α,ω-dihydroxypolybutadiene.

The mixture comprising the base polyesterol and the at least one further reagent subsequently flows through a reactor in which at least one packing comprising at least one immobilized enzyme on a support is present.

The enzyme acts as catalyst for the reaction of the base polyesterol with the at least one further reagent to form the polyesterol which is different from the base polyesterol.

Suitable enzymes which can be used as catalysts are preferably lipases or hydrolases. Preference is given to using a lipase, in particular one of the lipases Candida antarctica, Candida cylinderacea, Mucor miehei, Pseudomonas cepacia, Pseudomonas fluorescens and Burkholderia plantarii. The temperature at which the reactor for preparing the polyesterol from the at least one base polyesterol and the at least one further reagent is operated is preferably in the range from 50 to 110° C., more preferably in the range from 50 to 90° C. The pressure at which the reactor is operated is preferably in the range from 0.5 to 10 bar, more preferably in the range from 0.5 to 5 bar.

For the reaction to be able to be carried out in a continuous reactor, it is necessary for the at least one enzyme to be immobilized on a support. As support materials, it is possible to use all suitable materials, but preferably solid materials having large surface areas, more preferably resins, polymers, etc., on which the enzymes can preferably be bound covalently. Suitable support materials are polyacrylate, polyacrylamide, polyamide, polystyrene, polypropylene, polyvinyl chloride, polyurethane, latex, nylon, Teflon, polypeptides, agarose, cellulose, dextran, silica, glass, ceramic, kieselguhr, for example Celite®, wood charcoal or wood black, sawdust, hydroxyapatite and aluminum. Particularly preferred support materials are polyacrylate, polyamide, polystyrene, silica, glass and ceramic.

It is possible to use, for example, resin beads having a small diameter as support material. Such resin beads are suitable, for example, for forming a moving bed, a fixed bed or a fluidized bed. Furthermore, it is also possible for the support to be present in the form of a packing or in the form of random packing elements. These are used, for example, when the reactor comprises a structured or unstructured packing on which the enzyme is bound. Preferred supports are silica gel, aluminum oxide, molecular sieves, anionic and cationic ion exchange resins.

Particularly in the case of reactors in which the enzyme immobilized on the support material is present as a moving bed, fixed bed, random particulate material or fluidized bed, it is possible for part of the immobilized enzyme to be entrained in the medium flowing through and carried out of the reactor. In this case, the enzymes immobilized on the support material are preferably separated off from the polyesterol after passage through the reactor. This separation can, for example, be achieved by classical separation processes such as filtration, centrifugation or the like which exploit the differing particle size or the differing particle weight. The separation can, for example, also be effected by the use of magnetic forces in the case of magnetic support materials. The removal of the enzymes immobilized on support materials after passage through the reactor prevents these from interfering in the use of the polyesterols prepared, in particular in further reactions of these polyesterols, such as, for example, in the reaction of the polyesterols with isocyanates to form polyurethanes.

A reactor suitable for carrying out the process preferably comprises an inlet and an outlet and the reaction mixture flows through it continuously. At least one packing, a fixed bed or a fluidized bed comprising the enzyme immobilized on the support is present in the reactor. The free volume of the packing, the fixed bed or the fluidized bed based on the total volume of the packing, the fixed bed or the fluidized bed is preferably in the range from 10 to 100%. The ratio of the free volume of the packing, the fixed bed or the fluidized bed to the total volume of the packing, the fixed bed or the fluidized bed is more preferably in the range from 30 to 100% and in particular in the range from 50 to 100%. Furthermore, in the packing comprising the immobilized enzyme, the ratio of the free flow cross section to the cross section of the packing is preferably in the range from 10 to 80%, more preferably in the range from 30 to 78% and in particular in the range from 50 to 74%.

To carry out the reaction of the at least one base polyesterol with the at least one further reagent, it is necessary for these to be mixed. It is possible for the at least one base polyesterol and the at least one further reagent to be introduced separately into the reactor and be mixed in the reactor or else for the at least one base polyesterol and the at least one further reagent to be mixed before introduction into the reactor.

If the at least one base polyesterol and the at least one further reagent are mixed before introduction into the reactor, mixing is preferably carried out in a mixing apparatus of one of the types known to those skilled in the art. It is possible to use, for example, customary static or dynamic mixers for this purpose. Such static mixers comprise, for example, internals which deflect the flow and thereby generate turbulence by means of which the reagents are mixed. In contrast, dynamic mixers comprise moving parts, for example rotors or stirrers.

The reaction mixture, when the at least one base polyesterol and the at least one further reagent are mixed before introduction into the reactor, or the reagents, when mixing occurs in the reactor, are preferably introduced at the bottom of the reactor. As a result of this, uniform flow is achieved immediately on start-up when the reactor is not yet full of liquid.

Measurements of the reaction kinetics of the enzymatic transesterification have shown that long residence times or high catalyst concentrations are necessary to carry out the transesterification reaction. The residence time required for the reaction can, for example, be achieved by the reaction mixture passing through the reactor a number of times. Furthermore, it is also possible to select the flow rate of the reaction mixture so that the time taken for the reaction mixture to flow through the packing comprising the immobilized enzyme corresponds to the required reaction time.

The reaction of the at least one base polyesterol and the at least one further reagent to form the polyesterol can be carried out in the presence of a solvent. If the reaction is carried out in the presence of a solvent, it is possible to use all known suitable solvents, in particular the solvents toluene, dioxane, hexane, tetrahydrofuran, cyclohexane, xylene, dimethyl sulfoxide, dimethylformamide, N-methylpyrrolidone, chloroform. The choice of solvent depends on the starting materials (the at least one base polyesterol and the at least one further reagent) used in the particular case and, in particular, on their solubility properties. However, the reaction in the presence of a solvent has the disadvantage that additional process substeps, namely the dissolution of the at least one base polyesterol in the solvent and the removal of the solvent after the reaction, become necessary. Furthermore, the dissolution of the at least one base polyesterol in the solvent can, depending on the hydrophobicity properties of the base polyesterol, be problematical and may decrease the yield.

In a preferred embodiment of the process, the reaction of the at least one base polyesterol and the at least one further reagent is carried out in the absence of a solvent (also referred to as “reaction in bulk”). If base polyesterols having a high molecular weight are to be subjected to the enzymatic transesterification, the effectiveness of this transesterification reaction is limited by the low solubility of these base polyesterols of high molecular weight in most solvents. On the other hand, the number of hydroxyl groups of the solvent has only a small influence on the effectiveness of the transesterification reaction. Thus, for example, according to McCabe and Taylor, Tetrahedron 60 (2004), 765 to 770, no transesterification reaction takes place in 1,4-butanediol as solvent even though the concentration of hydroxyl groups is very high. In contrast, transesterification does take place in polar solvents (dioxane, toluene).

In a further preferred embodiment of the process, preference is given to using base polyesterols, enzymes and, if appropriate, additional polyhydroxyl compounds which together have a water content of less than 0.1% by weight, preferably less than 0.05% by weight, more preferably less than 0.03% by weight, in particular less than 0.01% by weight, for preparing the polyesterol. In the case of higher water contents, hydrolysis also takes place alongside the transesterification during the reaction of the at least one base polyesterol with the at least one further reagent, so that the acid number of the polyesterol would increase in an undesirable way during the transesterification. Carrying out the transesterification of the process of the invention at a water content of less than 0.1% by weight, preferably less than 0.05% by weight, more preferably less than 0.03% by weight, in particular less than 0.01% by weight, thus leads to the preparation of special polyesterols having a low acid number as end products.

Polyesterols having a low acid number are generally more stable toward hydrolysis than polyesterols having a high acid number, since free acid groups catalyze the reverse reaction, i.e. the hydrolysis.

Preparation of polyesterols having water contents above 0.1% by weight leads to polyesterols having an acid number of greater than 10 mg KOH/g. However, polyesterols having such high acid numbers (>10 mg KOH/g) are unsuitable or have only poor suitability for most industrial applications, in particular for use in the preparation of polyesterols.

Depending on the atmospheric humidity, enzymes can have water contents of >0.1% by weight. For this reason, drying of the enzyme is necessary before use of the enzyme in the transesterification reaction. Drying of the enzyme is carried out by the customary drying methods, e.g. by drying in a vacuum drying oven at temperatures of from 60 to 120° C. under a pressure of from 0.5 to 100 mbar or by suspending the enzyme in toluene and subsequently distilling off the toluene under reduced pressure at temperatures of from 50 to 100° C.

Polyesterols, too, take up at least 0.01%, but generally at least 0.02%, in some cases even more than 0.05%, by weight of water, depending on the atmospheric humidity and temperature. Depending on the degree of conversion and molecular weight of the base polyesterols used, this water concentration is higher than the equilibrium water concentration. If the base polyesterol is not dried before the transesterification, hydrolysis of the polyesterol inevitably occurs.

The base polyesterols used for the transesterification are therefore preferably dried prior to the transesterification. The enzyme to be used and the at least one further reagent are also preferably dried prior to the transesterification reaction in order to achieve the abovementioned low water content in the transesterification. Drying can be carried out using customary drying methods of the prior art, for example by drying over molecular sieves or by means of a falling film evaporator. As an alternative, base polyesterols having low water contents (preferably less than 0.1% by weight, more preferably less than 0.05% by weight, even more preferably less than 0.03% by weight, in particular less than 0.01% by weight) can also be obtained by carrying out the reaction and also any intermediate storage of the at least one base polyesterol entirely under inert conditions, for example in an inert gas atmosphere, preferably in a nitrogen atmosphere. In this case, the base polyesterols have no opportunity of taking up relatively large amounts of water from the environment right from the beginning. A separate drying step could then become superfluous.

Customary types of reactor which can be used for carrying out the transesterification according to the invention are, for example, columns which comprise a structured or unstructured packing, moving-bed reactors or fluidized-bed reactors. The material of which the reactor is constructed has to be resistant to corrosion, heat and acid. Suitable materials are, for example, stainless steel, glass and ceramic. Suitable stainless steels are, for example, austenitic chromium-nickel-molybdenum alloys (for example V4A steel DIN 1.4571).

The invention is illustrated below with the aid of an example.

### EXAMPLE

The base polyesterol polyethylene glycol adipate is placed in a heated stirred vessel. Diethylene glycol is added to this while stirring in order to obtain the desired acid number of 150 mg KOH/g. The mixture is subsequently introduced into a reaction column comprising a packing of Novozym 435®. This is the commercially available, immobilized form of lipase B from Candida antarctica. In the reactor, the reaction product obtained by transesterification in the reactor is conveyed into a collection vessel. Samples to determine the viscosity and the GPC are taken from the column at regular intervals. The flow rates are in the range from 860 to 1000 g/h. To test the life of the enzyme, a total amount of 95 kg of the mixture of polydiethylene glycol adipate and diethylene glycol is fed to the column over a period of 5 days.

The column used has a diameter of 30 mm and a length of 1 m and is made of glass. The volume of the column is 700 ml. The column is charged with 180 g of the dried enzyme which has been dissolved in polyesterol. The concentration of Novozym® is 25% by weight. A higher degree of filling is not possible because of the swelling of the catalyst and the increasing pressure drop resulting therefrom.

1. A process for preparing polyesterols which are different from at least one base polyesterol from the at least one base polyesterol and at least one further reagent, wherein the process is performed continuously and wherein
(a) the at least one base polyesterol and the at least one further reagent are mixed,
(b) the mixture produced in (a) flows through a reactor in which at least one packing comprising at least one immobilized enzyme on a support is present, with the at least one base polyesterol and the at least one further reagent being reacted to form the polyesterol.

2. The process according to claim 1, wherein the at least one base polyesterol and the at least one further reagent are introduced separately into the reactor and mixed in the reactor.

3. The process according to claim 1, wherein the at least one base polyesterol and the at least one further reagent are mixed before introduction into the reactor.

4. The process according to claim 1, wherein the further reagent is a polyesterol, a polyol, an organic acid or an oligomer or polymer having at least one hydroxyl or carboxylic acid radical.

5. The process according to claim 1, wherein the reactor is operated at a temperature in the range from 50 to 120° C. and a pressure in the range from 0.5 to 10 bar.

6. The process according to claim 1, wherein, in the packing comprising the immobilized enzyme, the ratio of the free flow cross section to the cross section of the packing is in the range from 10 to 80%.

7. The process according to claim 1, wherein the at least one enzyme is a lipase or a hydrolase.

8. The process according to claim 7, wherein the lipase is selected from among Candida antarctica, Candida cylinderacea, Mucor miehei, Pseudomonas cepacia, Pseudomonas fluorescens and Burkholderia plantarii.

9. An apparatus for carrying out the process according to claim 1 which comprises a reactor which has an inlet and an outlet and through which the reaction mixture flows continuously and in which at least one packing, a fixed bed or a fluidized bed comprising the enzyme immobilized on the support is present.

10. The apparatus according to claim 9, wherein the reaction mixture or the reagents are introduced at the bottom of the reactor.

11. The apparatus according to claim 9, wherein the free volume of the packing, the fixed bed or the fluidized bed based on the total volume of the packing, the fixed bed or the fluidized bed is in the range from 10 to 100%.

12. The apparatus according to claim 9, wherein the support material on which the enzyme is immobilized is polyacrylate, polyamide, polystyrene, silica, glass, ceramic.

13. The apparatus according to claim 9, wherein the reactor is made of stainless steel, glass or ceramic.
